(19) United States
(12) Patent Application Publication
(10) Pub. No.: US 2023/0277666 A1
(43) Pub. Date: Sep. 7, 2023

(54) CHIMERIC ANTIGEN RECEPTOR COMPRISING CO-STIMULATORY RECEPTOR AND APPLICATION THEREOF

(71) Applicant: SHANGHAI LONGYAO BIOTECHNOLOGY INC., LTD., Shanghai (CN)

(72) Inventors: Xuanming YANG, Shanghai (CN); Yangxin FU, Shanghai (CN); Xin WANG, Shanghai (CN); Shengqin YE, Shanghai (CN); Fanlin LI, Shanghai (CN); Huihui ZHANG, Shanghai (CN)

(21) Appl. No.: 18/159,887

(22) Filed: Jan. 26, 2023

Related U.S. Application Data
(63) Continuation-in-part of application No. 17/126,966, filed on Dec. 18, 2020, now Pat. No. 11,590,168.
Application No. 17/126,966 is a continuation of application No. PCT/CN2019/077922, filed on Mar. 13, 2019.

(30) Foreign Application Priority Data
Jun. 20, 2018 (CN) 201810636409.0

(51) Int. Cl. A61K 39/00; C07K 14/725; C07K 16/28; C07K 14/705; A61P 35/00

(52) U.S. Cl. CPC A61K 39/4611; C07K 14/7051; C07K 16/2887; C07K 14/70578; C07K 14/70521; A61P 35/00; A61K 39/4631; A61K 39/4643; C07K 2317/622; A61K 2239/13; A61K 2239/22

(57) ABSTRACT

Provided by the present invention is a chimeric antigen receptor comprising a co-stimulatory receptor, the chimeric antigen receptor having a structure of scFv(X)-(Y)CD3zeta-2A-(Z); X comprises a tumor targeting antibody or a ligand or receptor capable of specifically binding to a tumor; Y is an intracellular region of the co-stimulatory receptor, and Z is a co-stimulatory receptor that is selected from among ICOS, CD28, CD27, HVEM, LIGHT, CD40L, 4-1BB, OX40, DR3, GITR, CD30, TIMI, SLAM, CD2, CD226. Further provided by the present invention are CAR-T cells that are constructed by means of a recombinant expression vector of the described chimeric antigen receptor, a preparation method therefor and an application thereof. The CAR-T cells described in the present invention significantly improve the tumor-killing abilities and amplification abilities thereof.

The present application is a continuation-in-part of U.S. patent application Ser. No. 17/126,966, filed Mar. 13, 2019, a continuation of PCT/CN2019/077922, filed Mar. 13, 2019, which claims the benefit of CN application 2018106364090 (CN), filed Jun. 20, 2018, all of which are hereby incorporated by reference in their entireties herein.

### SEQUENCE LISTING

This application incorporates in its entirety the Sequence Listing entitled “271302-521213SequenceListing.xml” (17,360 bytes), which was created on Jan. 19, 2023, and filed electronically herewith.

### TECHNICAL FIELD

The present invention relates to the field of cellular immunotherapeutic technology, especially relates to a chimeric antigen receptor comprising a co-stimulatory receptor and use thereof.

### BACKGROUND OF THE INVENTION

The use of immunological therapy for overcoming tumors has always been an important direction in the application of immunology in translational medicine. With the development of various omics (genomics, proteomics, etc.), tumor cells have been widely recognized due to their immunogenicity caused by mutations, which lays a theoretical foundation for tumor immunotherapy. At the same time, with the accumulation of tumor immunology research itself, tumor immunotherapy has recently made a great progress, and a series of new immunotherapy methods have gradually entered into the clinic. The current tumor immunology research has established the central position of T cell killing in tumor immunotherapy, and the chimeric antigen receptor T cell (CAR-T cell) is a tumor-killing cell which has combined the targeted recognition of antibody and the tumor-killing function of T cell, and been generated by artificial modification.

The concept of chimeric antigen receptor T cell was first proposed by Gross, Waks and Eshhar in 1989. They expressed TNP-recognizing antibodies on T cells, achieving antigen-specific, non-MHC-restricted T cell activation and enhanced effect, and proposed the concept of the application of CAR-T technology in tumor treatment. According to this principle, tumor-specific antibodies are embedded into T cells, which will give T cells new tumor-killing capabilities. After that, CAR-T technology was introduced into anti-tumor clinical trials, but the final clinical results of early CAR-T cells are not ideal since their intracellular signal transmission domain contains only the first signal, and the selected tumor type is a solid tumor. In 2008, the Fred Hutchison Cancer Institute and other institutions used CAR-T to treat B cell lymphoma, although the treatment results are not ideal, the key to this clinical trial is to demonstrate that CAR-T treatment with CD20-expressing B cells as the target is relatively safe. Subsequently, in 2010, NCI reported a case of successful treatment of B-cell lymphoma, using CAR-T targeting CD19, the patient's lymphoma was controlled, normal B cells were also eliminated, and serum Ig was significantly reduced, providing a theoretical and practical support for the effectiveness of CAR-T in the treatment of B cell-derived lymphomas. In 2011, a team led by Dr. Carl June of the University of Pennsylvania in the United States used CAR-T that specifically recognizes CD19 for the treatment of chronic lymphocytic leukemia derived from B cells, showing a “cure” effect. After that, clinical trials have also been launched in relapsed and refractory acute lymphoblastic cell leukemia, and good results have also been achieved. Due to this breakthrough progress and the development of other immune regulation methods, Science magazine ranked tumor immunotherapy as the number one scientific and technological breakthrough in 2013. This success has caused widespread influence in countries around the world, and countries have begun to carry out a large number of CAR-T-based scientific research and clinical trials of tumor treatment.

The structure of CAR consists of an extracellular antigen recognition domain, an extracellular hinge region, a transmembrane domain, and an intracellular signal transduction domain. The extracellular antigen recognition domain generally consists of a single-chain antibody, which specifically recognizes membrane surface molecules of the tumor cell, or can be a ligand or receptor of certain tumor-specific antigens, etc. The extracellular hinge region is a spatial structure that separates the antigen recognition domain from the transmembrane domain, and its purpose is to provide a suitable spatial position, so that the extracellular antigen recognition domain can maintain the correct structure and transmit the intracellular signals before and after recognizing the antigen. The transmembrane domain is a domain for ensuring the positioning of the CAR molecule on the membrane surface. The intracellular signal transduction domain is a key part of mediating the CAR signal transduction, and is usually a combination of one or several first signals (for the recognition of TCR and WIC-I-peptide complex) and second signals (for the recognition of costimulatory receptor and costimulatory ligand). The first-generation CAR contains only the first signal, the second-generation CAR has one first signal and one second signal, and the third-generation CAR has one first signal and two second signal domains. Although CAR-T has achieved a great success in the treatment of leukemia derived from B cell, its relatively high recurrence rate and low effectiveness for solid tumors are important challenges currently. Therefore, there is an urgent clinic need of developing a new generation of high-efficiency CAR-T currently. In addition to the third-generation CAR-T, there are currently other new CAR-T design strategies, that is, new regulatory molecules independent of CAR are introduced on the basis of the second-generation CAR-T to further enhance the function of CAR-T.

The application of CAR-T targeting the B cell surface targeting molecules CD19 and CD20 prepared from the patient's own blood cells in the treatment of B cell leukemia has been relatively mature, but there are a large number of recurrences, even though the response rate is high. In addition, the treatment efficiency for solid lymphoma is relatively low, which is related to the immunosuppressive microenvironment in solid tumors.

In solid tumors, there are a variety of immune cells, tumor cells and stromal cells, which together constitute the tumor microenvironment. The tumor microenvironment is usually immunosuppressive, and can inhibit endogenous anti-tumor T cell responses or adoptive T cells (such as CAR-T) at multiple levels, for example, leading to exhaustion of T cells and loss of tumor killing function, and eventually leading to the clearance of T cells. How to enhance the activation ability of CAR-T in solid tumors so that CAR-T can fight against the immune suppression in the tumor microenvironment is an important idea and direction for expanding CAR-T to solid tumor treatment.

However, the current CAR-T domains in clinical use still have insufficient tumor killing and expansion abilities, and have poor efficacy in controlling solid tumors/metastasis. Some CAR-T use novel regulatory molecules such as IL-12, 4-1BBL, etc. These molecules will also produce non-specific activation effects on other non-CAR-T cells in addition to affecting the CAR-T, which may cause immune side effects.

### SUMMARY OF THE INVENTION

An object of the present invention is to address the defects in the prior art, provide a chimeric antigen receptor including a co-stimulatory receptor and use thereof, and provide a CAR-T cell constructed by a recombinant expression vector of the chimeric antigen receptor. For example, OX40 is an important co-stimulatory receptor which is primarily expressed in activated CD4 and CD8 T cells, and displays a variety of functions during the activation of T cells. They can promote the activation of T cells, exhibit more effector molecules, and reduce the expression of gene associated with apoptosis. Integrating the co-stimulatory receptor signal into the CAR-T has a potential effect-enhancing function.

To address the aforesaid object, the present invention utilizes the following technical solutions:

a first object of the present invention is to provide a chimeric antigen receptor including a co-stimulatory receptor and having a structure of scFv(X)-(Y)CD3zeta-2A-(Z); wherein X is a tumor-targeting antibody or a ligand or receptor capable of specifically binding to a tumor; Y is an intracellular domain of a co-stimulatory receptor, and said co-stimulatory receptor is selected from a group consisting of ICOS, CD28, CD27, HVEM, LIGHT, CD40L, 4-1BB, OX40, DR3, GITR, CD30, TIM1, SLAM, CD2, and CD226; Z is a co-stimulatory receptor, and said co-stimulatory receptor is selected from a group consisting of ICOS, CD28, CD27, HVEM, LIGHT, CD40L, 4-1BB, OX40, DR3, GITR, CD30, TIMI, SLAM, CD2, and CD226.

For further optimizing the aforesaid chimeric antigen receptor, the technical means used in the present invention further includes:

Further, the X is selected from a group consisting of anti-CD19 antibody, anti-CD20 antibody, EGFR antibody, HER2 antibody, EGFRVIII antibody, anti-PSMA antibody, anti-BCMA antibody, anti-CD22 antibody, and anti-CD30 antibody. Understandably, X can also be other protein capable of specifically binding to a tumor.

Further, said X is anti-CD20 antibody, anti-CD19 antibody or anti-EGFR antibody.

In some embodiments, said Y is an intracellular domain of 4-1BB.

In some embodiments, said Z is one selected from a group consisting of OX40, HVEM, ICOS, CD27, and 4-1BB. In some embodiments, said Z is OX40. In some embodiments, said Z is ICOS. In some embodiments, said Z is CD27.

In some embodiments, said antibody comprises scFv.

In some embodiments, said X is selected from a group consisting of anti-CD19 scFv, anti-CD20 scFv, anti-EGFR scFv, anti-HER2 scFv, anti-EGFRVIII scFv, anti-PSMA scFv, anti-BCMA scFv, anti-CD22 scFv, and anti-CD30 scFv. In some embodiments, said X is selected from a group consisting of anti-CD19 scFv, anti-CD20 scFv, and anti-EGFR scFv.

Further, the sequence of said anti-CD20 scFv is as set forth in SEQ ID No: 1; the sequence of said anti-CD19 scFv is as set forth in SEQ ID No: 11; and/or the sequence of said anti-EGFR scFv is as set forth in SEQ ID No: 12.

Further, said OX40 has a sequence of SEQ ID No.2; said HVEM has a sequence of SEQ ID No.3; said ICOS has a sequence of SEQ ID No.4; said CD27 has a sequence of SEQ ID No.5; and/or said 4-1BB has a sequence of SEQ ID No.6.

Further, said 2A has a sequence of SEQ ID No.7, SEQ ID No.8, SEQ ID No.9 or SEQ ID No.10.

In some embodiments, said X is anti-CD20 scFv, and said Y is an intracellular domain of 4-1BB, said Z is one selected from a group consisting of OX40, HVEM, ICOS, CD27, and 4-1BB. In some embodiments, said X is anti-CD20 scFv, and said Y is an intracellular domain of 4-1BB, said Z is OX40. In some embodiments, said X is anti-CD20 scFv, and said Y is an intracellular domain of 4-1BB, said Z is CD27. In some embodiments, said X is anti-CD20 scFv, and said Y is an intracellular domain of 4-1BB, said Z is ICOS.

In some embodiments, said X is anti-CD19 scFv, and said Y is an intracellular domain of 4-1BB, said Z is one selected from a group consisting of OX40, HVEM, ICOS, CD27, and 4-1BB. In some embodiments, said X is anti-CD19 scFv, and said Y is an intracellular domain of 4-1BB, said Z is OX40. In some embodiments, said X is anti-CD19 scFv, and said Y is an intracellular domain of 4-1BB, said Z is CD27. In some embodiments, said X is anti-CD19 scFv, and said Y is an intracellular domain of 4-1BB, said Z is ICOS.

In some embodiments, said X is anti-EGFR scFv, and said Y is an intracellular domain of 4-1BB, said Z is one selected from a group consisting of OX40, HVEM, ICOS, CD27, and 4-1BB. In some embodiments, said X is anti-EGFR scFv, and said Y is an intracellular domain of 4-1BB, said Z is OX40. In some embodiments, said X is anti-EGFR scFv, and said Y is an intracellular domain of 4-1BB, said Z is CD27. In some embodiments, said X is anti-EGFR scFv, and said Y is an intracellular domain of 4-1BB, said Z is ICOS.

Wherein the aforesaid sequences are as follows:

SEQ ID No. 1:
QIVLSQSPAILSASPGEKVTMTCRASSSVSYIHWFQQKPGSSPKPWIYAT
SNLASGVPVRFSGSGSGTSYSLTISRVEAEDAATYYCQQWTSNPPTFGGG
TKLEIKGGGGSGGGGSGGGGSQVQLQQPGAELVKPGASVKMSCKASGYTF
TSYNMHWVKQTPGRGLEWIGAIYPGNGDTSYNQKFKGKATLTADKSSSTA
YMQLSSLTSEDSAVYYCARSTYYGGDWYFNVWGAGTTVTVSAAAATTTPA
PRPPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEE
EGGCELRVKFSRSADAPAYQQGQNQLYNELNLGRREEYDVLDKRRGRDPE
MGGKPRRKNPQEGLYNELQKDKMAEAYSEIGMKGERRRGKGHDGLYQGLS
TATKDTYDALHMQALPPR;
SEQ ID No. 2:
MCVGARRLGRGPCAALLLLGLGLSTVTGLHCVGDTYPSNDRCCHECRPGN
GMVSRCSRSQNTVCRPCGPGFYNDVVSSKPCKPCTWCNLRSGSERKQLCT
ATQDTVCRCRAGTQPLDSYKPGVDCAPCPPGHFSPGDNQACKPWTNCTLA
GKHTLQPASNSSDAICEDRDPPATQPQETQGPPARPITVQPTEAWPRTSQ
GPSTRPVEVPGGRAVAAILGLGLVLGLLGPLAILLALYLLRRDQRLPPDA
HKPPGGGSFRTPIQEEQADAHSTLAKI;
SEQ ID No. 3:
MEPPGDWGPPPWRSTPKTDVLRLVLYLTFLGAPCYAPALPSCKEDEYPVG
SECCPKCSPGYRVKEACGELTGTVCEPCPPGTYIAHLNGLSKCLQCQMCD
PAMGLRASRNCSRTENAVCGCSPGHFCIVQDGDHCAACRAYATSSPGQRV
QKGGTESQDTLCQNCPPGTFSPNGTLEECQHQTKCSWLVTKAGAGTSSSH
WVWWFLSGSLVIVIVCSTVGLIICVKRRKPRGDVVKVIVSVQRKRQEAEG
EATVIEALQAPPDVTTVAVEETIPSFTGRSPNH;
SEQ ID No. 4:
MKSGLWYFFLFCLRIKVLTGEINGSANYEMFIFHNGGVQILCKYPDIVQQ
FKMQLLKGGQILCDLTKTKGSGNTVSIKSLKFCHSQLSNNSVSFFLYNLD
HSHANYYFCNLSIFDPPPFKVTLTGGYLHIYESQLCCQLKFWLPIGCAAF
VWCILGCILICWLTKKKYSSSVHDPNGEYMFMRAVNTAKKSRLTDVTL;
SEQ ID No. 5:
MARPHPWWLCVLGTLVGLSATPAPKSCPERHYWAQGKLCCQMCEPGTFLV
KDCDQHRKAAQCDPCIPGVSFSPDHHTRPHCESCRHCNSGLLVRNCTITA
NAECACRNGWQCRDKECTECDPLPNPSLTARSSQALSPHPQPTHLPYVSE
MLEARTAGHMQTLADFRQLPARTLSTHWPPQRSLCSSDFIRILVIFSGMF
LVFTLAGALFLHQRRKYRSNKGESPVEPAEPCRYSCPREEEGSTIPIQED
YRKPEPACSP;
SEQ ID No. 6:
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCEL;
SEQ ID No. 7:
GSGATNFSLLKQAGDVEENPGP;
SEQ ID No. 8:
GSGEGRGSLLTCGDVEENPGP;
SEQ ID No. 9:
GSGQCTNYALLKLAGDVESNPGP;
SEQ ID No. 10:
GSGVKQTLNFDLLKLAGDVESNPGP.
SEQ ID No. 11:
METDTLLLWVLLLWVPGSTGTGDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITGGGGSGGGGSGGGGSEVKLQE
SGPGLVAPSQSLSVTCTVSGVSLPDYGVSWIRQPPRKGLEWLGVIWGSET
TYYNSALKSRLTIIKDNSKSQVFLKMNSLQTDDTAIYYCAKHYYYGGSYA
MDYWGQGTSVTVSS
SEQ ID No. 12:
METDTLLLWVLLLWVPGSTGTGDILLTQSPVILSVSPGERVSFSCRASQS
IGTNIHWYQQRTNGSPRLLIKYASESISGIPSRFSGSGSGTDFTLSINSV
ESEDIADYYCQQNNNWPTTFGAGTKLELKGGGGSGGGGSGGGGSQVQLKQ
SGPGLVQPSQSLSITCTVSGFSLTNYGVHWVRQSPGKGLEWLGVIWSGGN
TDYNTPFTSRLSINKDNSKSQVFFKMNSLQSNDTAIYYCARALTYYDYEF
AYWGQGTLVTVSA

Further, the extracellular hinge region of said chimeric antigen receptor is a region selected from CD8a or IgG; and the transmembrane domain of said chimeric antigen receptor is one selected from a group consisting of CD8a, CD28, CD137 and CD3.

A second object of the present invention is to provide a recombinant expression vector of any one of the aforesaid chimeric antigen receptors.

In some embodiments, the vector comprises DNA and RNA.

In some embodiments, the vector is selected from the group consisting of plasmid, virus vector, transposon, and a combination thereof.

In some embodiments, the vector comprises a DNA virus and a retrovirus vector.

In some embodiments, the vector is selected from the group consisting of a lentiviral vector, an adenovirus vector, an adeno-associated virus vector, and a combination thereof.

In some embodiments, the vector is a lentiviral vector.

A third object of the present invention is to provide a CAR-T cell constructed by a recombinant expression vector of any one of the aforesaid chimeric antigen receptors.

In some embodiments, the cell is an isolated cell.

In some embodiments, the cell is a genetically engineered cell.

In some embodiments, the cell is a mammalian cell.

In some embodiments, the cell is a CAR-T cell and/or a CAR-NK cell.

The instant invention also provides a chimeric antigen receptor-T (CAR-T) cell comprising an expression vector encoding and expressing the CAR as described in the present application.

The instant invention also provides a method of preventing or treating a tumor, comprising administrating said CAR-T cell as described in the present application to a subject in need thereof.

A fourth object of the present invention is to provide a method of preparing the aforesaid CAR-T cell which includes the following steps:

step 1: construction of lentiviral vector and production of virus;

incorporating 2A between scFv(X)-(Y)CD3zeta and Z to form a fusion protein, adding a lentiviral vector to both ends of the fusion protein, and co-transfecting with lentiviral packaging plasmid to obtain an scFv(X)-(Y)CD3zeta-2A-(Z) virus;

For further optimizing the method of preparing the aforesaid CAR-T cell, the technical means used in the present invention further includes: step 2, preparation of scFv(X)-(Y)CD3zeta-2A-(Z) CAR-T cell;

culturing purified human PBMC and infecting the T cell isolated from said PBMC with the scFv(X)-(Y)CD3zeta-2A-(Z) virus obtained in Step 1, and subjecting them to cell expansion under suitable conditions to prepare scFv(X)-(Y)CD3zeta-2A-(Z) CAR-T cell.

For further optimizing the method of preparing the aforesaid CAR-T cell, the technical means used in the present invention further includes:

Further, said construction of lentiviral vector and production of virus include: incorporating 2A between scFv(X)-(Y)CD3zeta and Z by overlap PCR to form a fusion protein, and adding restriction sites to both ends of the fusion protein to clone a lentiviral vector; subjecting the clones sequenced correctly to a large scale endotoxin-free extraction, and co-transfecting with lentiviral packaging plasmid, after a predetermined period of time, collecting a supernatant, filtering, centrifuging to concentrate the virus to obtain an scFv(X)-(Y)CD3zeta-2A-(Z) virus.

Still further, the specific steps of the construction of lentiviral vector and production of virus are as follows: incorporating 2A sequence between scFv(X)-(Y)CD3zeta and OX40 by overlap PCR, adding EcoRI and SalI restriction sites to both ends of the fusion protein to clone the pCDH-MSCVEF vector, subjecting the clones sequenced correctly to a large scale endotoxin-free extraction, and co-transfecting with lentiviral packaging plasmid into 293X; after 48 and 72 hours, collecting the supernatant, filtering it by a 0.45 μmfilter and performing centrifugation at 25000 RPM for 2 hours to concentrate the viruses to obtain the scFv(X)-(Y)CD3zeta-2A-(Z) virus.

Further, the specific steps of the preparation of scFv(X)-(Y)CD3zeta-2A-(Z) CAR-T cell include: isolating T cells from human PBMC for purification, incubating into a culture plate under suitable stimulation conditions, culturing for a predetermined period of time, infecting said T cells with the scFv(X)-(Y)CD3zeta-2A-(Z) virus obtained in Step 1, and subjecting it to cell expansion under suitable stimulation conditions, after 2 rounds of expansion under stimulation, the obtained cells are the scFv(X)-(Y)CD3zeta-2A-(Z) CAR-T cells.

Further, the stimulation conditions for culturing the isolated and purified human PBMC are anti-hCD3 antibody and anti-hCD28 antibody; and the stimulation conditions for cell expansion are stimulation by use of artificial antigen presenting cell or anti-hCD3/28 antibody every 6 days.

Still further, the specific steps of preparing the scFv(X)-(Y)CD3zeta-2A-(Z) CAR-T cell are as follows: purifying human PBMC by a Stemcell T cell isolation kit, inoculating into a 96-well culture plate coated by anti-hCD3 and anti-hCD28. After 2 days, infecting the T cells with the scFv(X)-(Y)CD3zeta-2A-(Z) virus at MOI=10-20. After 1 day, continuing to culture the cells with the medium changed, and stimulating them by artificial antigen presenting cell or anti-hCD3/28 every 6 days. After 2 rounds of stimulation, the obtained cells are scFv(X)-(Y)CD3zeta-2A-(Z) CAR-T cells.

Further, said X is selected a group consisting of anti-CD19 antibody, anti-CD20 antibody, EGFR antibody, HER2 antibody, EGFRVIII antibody, anti-PSMA antibody, anti-BCMA antibody, anti-CD22 antibody, and anti-CD30 antibody. Further, said X is anti-CD20 antibody, anti-CD19 antibody or EGFR antibody.

In some embodiments, said Y is an intracellular domain of 4-1BB.

In some embodiments, said Z is one selected from a group consisting of OX40, HVEM, ICOS, CD27, and 4-1BB. In some embodiments, said Z is OX40. In some embodiments, said Z is CD27. In some embodiments, said Z is ICOS.

Further, said X is anti-CD20 antibody, said Y is 4-1BB, said Z is one selected from a group consisting of OX40, HVEM, ICOS, CD27, and 4-1BB. Further, said Xis anti-CD20 antibody, said Y is 4-1BB, said Z is OX40. Further, said X is anti-CD20 antibody, said Y is 4-1BB, said Z is CD27. Further, said X is anti-CD20 antibody, said Y is 4-1BB, said Z is ICOS.

Further, said X is anti-CD19 antibody, said Y is 4-1BB, said Z is one selected from a group consisting of OX40, HVEM, ICOS, CD27, and 4-1BB. Further, said X is anti-CD20 antibody, said Y is 4-1BB, said Z is OX40.

Further, said X is anti-EGFR antibody, said Y is 4-1BB, said Z is one selected from a group consisting of OX40, HVEM, ICOS, CD27, and 4-1BB. Further, said X is anti-CD20 antibody, said Y is 4-1BB, said Z is OX40.

Further, said scFv(X)-(Y)CD3zeta is anti-CD20 scFv having a sequence of SEQ ID No. 1; said OX40 has a sequence of SEQ ID No.2; said HVEM has a sequence of SEQ ID No.3; said ICOS has a sequence of SEQ ID No.4; said CD27 has a sequence of SEQ ID No.5; said 4-1BB has a sequence of SEQ ID No.6; and/or, said 2A has a sequence of SEQ ID No.7.

Further, said scFv(X)-(Y)CD3zeta is anti-CD19 scFv having a sequence of SEQ ID No. 11; said OX40 has a sequence of SEQ ID No.2; said HVEM has a sequence of SEQ ID No.3; said ICOS has a sequence of SEQ ID No.4; said CD27 has a sequence of SEQ ID No.5; said 4-1BB has a sequence of SEQ ID No.6; and/or, said 2A has a sequence of SEQ ID No.7.

Further, said scFv(X)-(Y)CD3zeta is anti-EGFR scFv having a sequence of SEQ ID No. 12; said OX40 has a sequence of SEQ ID No.2; said HVEM has a sequence of SEQ ID No.3; said ICOS has a sequence of SEQ ID No.4; said CD27 has a sequence of SEQ ID No.5; said 4-1BB has a sequence of SEQ ID No.6; and/or, said 2A has a sequence of SEQ ID No.7.

Further, the lentiviral packaging plasmid in Step 1 includes VSV-g, pMD Gag/Pol, RSV-REV, and the centrifugation is performed with Beckman ultracentrifuge and SW28 head.

A fifth object of the present invention is to provide a formulation including the aforesaid CAR-T cell or the CAR-T cell prepared by the aforesaid preparation method. Further, the formulation also includes a pharmaceutically diluents or excipient.

In some embodiments, the formulation is a liquid preparation.

In some embodiments, the formulation is an injection.

In some embodiments, the formulation comprises the host cell of the third aspect of the invention, and the concentration of the host cell is 1×10³−1×10⁸ cells/ml, preferably 1×10⁴−1×10⁷ cells/ml.

A sixth object of the present invention is to provide a use of the aforesaid chimeric antigen receptor, the aforesaid CAR-T cell or the CAR-T cell prepared by the aforesaid preparation method in preparation of a medicament for treating or preventing tumor.

A seventh object of the present invention is to provide a method of treating or preventing tumors, comprising administrating the aforesaid chimeric antigen receptor, the aforesaid CAR-T cell or the CAR-T cell prepared by the aforesaid preparation method to the subject in need of.

Further, said tumors are solid tumors. Examples of said solid tumors include, but are not limited to, lymphomas, renal tumors, neuroblastoma, germ cell tumor, osteosarcoma, chondrosarcoma, soft tissue sarcoma, liver tumor, thymoma, pulmonary blastoma, pancreato blastoma, hemangioma, etc.

In some embodiments, the CAR-T cell is administered intravenously, subcutaneously, intramuscularly, intraperitoneally, or through spinal.

In some embodiments, the CAR-T cell is administered intravenously.

In some embodiments, the CAR-T cell is allogeneic or autologous.

In some embodiments, wherein the subject is a human. Further, the subject in need of may be a subject having tumors.

In some embodiments, the tumor is selected from the group consisting of a hematological tumor, a solid tumor, and a combination thereof.

In some embodiments, the blood tumor is selected from the group consisting of Burkitt lymphoma (BL), acute myeloid leukemia (AML), multiple myeloma (MM), chronic lymphocytic leukemia (CLL), acute lymphoblastic leukemia (ALL), diffuse large B cell lymphoma (DLBCL), and a combination thereof.

In some embodiments, the tumor comprises Burkitt lymphoma (BL). In some embodiments, the tumor comprises acute lymphoblastic leukemia (ALL).

In some embodiments, the solid tumor is selected from the group consisting of gastric cancer, peritoneal metastasis of gastric cancer, liver cancer, leukemia, renal cancer, lung cancer, small intestine cancer, bone cancer, prostate cancer, colorectal cancer, breast cancer, large intestine cancer, cervical cancer, ovarian cancer, lymphoma, nasopharyngeal carcinoma, adrenal tumor, bladder tumor, non-small cell lung cancer (NSCLC), glioma, endometrial cancer, and a combination thereof.

In some embodiments, the solid tumor comprises lung cancer.

As compared with the prior art, the present invention has the following beneficial effects:

the CAR-T cell of the present invention significantly increases the tumor killing ability and expansion ability, and exhibits a greatly increased solid/metastasis tumor killing ability. The CAR-T cell of the present invention includes a co-stimulatory receptor (ICOS, CD28, CD27, HVEM, LIGHT, CD40L, 4-1BB, OX40, DR3, GITR, CD30, TIM1, SLAM, CD2, CD226, etc.), instead of a conventionally used ligand or excreted factor, and works only on the CAR-T cell, thereby reducing the risk of causing an immune side effect.

The present invention first utilizes the co-stimulatory receptor in the construction of CAR-T. As compared with the current CAR-T technology in clinic use, the present invention significantly increases the activation ability and survival ability of CAR-T cell in tumors, and controls the ability of solid/metastatic tumors, thereby improving the therapeutic effect of the CAR-T cell to get a more superior anti-tumor therapeutic effect.

### BRIEF DESCRIPTION OF THE DRAWINGS

FIG. 1 is an illustrative schematic view showing the molecular structure of chimeric antigen receptor (CAR) including the third signal receptor in embodiments of the present invention;

FIG. 2 is a schematic view showing the virus titer measured after 293 cells were infected with BBZ-2A-OX40 virus in an embodiment of the present invention;

FIG. 3 is a schematic view showing the virus titer measured after 293 cells were infected with BBZ-2A-HVEM virus in an embodiment of the present invention;

FIG. 4 is a schematic view showing the virus titer measured after 293 cells were infected with BBZ-2A-ICOS virus in an embodiment of the present invention;

FIG. 5 is a schematic view showing the virus titer measured after 293 cells were infected with BBZ-2A-CD27 virus in an embodiment of the present invention;

FIG. 6 is a schematic view showing the virus titer measured after 293 cells were infected with BBZ-2A-4-1BB virus in an embodiment of the present invention;

FIG. 7 is a schematic view showing the results of phenotypic analysis of BBZ CAR-T cell and BBZ-2A-OX40 CAR-T cell in an embodiment of the present invention;

FIG. 8 is a schematic view showing the results of phenotypic analysis of BBZ CAR-T cell and BBZ-2A-HVEM CAR-T cell in an embodiment of the present invention;

FIG. 9 is a schematic view showing the results of phenotypic analysis of BBZ CAR-T cell and BBZ-2A-ICOS CAR-T cell in an embodiment of the present invention;

FIG. 10 is a schematic view showing the results of phenotypic analysis of BBZ CAR-T cell and BBZ-2A-CD27 CAR-T cell in an embodiment of the present invention;

FIG. 11 is a schematic view showing the results of phenotypic analysis of BBZ CAR-T cell and BBZ-2A-4-1BB CAR-T cell in an embodiment of the present invention;

FIG. 12 is a schematic view showing the expansion ability of BBZ CAR-T cell and BBZ-2A-OX40 CAR-T cell in an embodiment of the present invention;

FIG. 13 is a schematic view showing the tumor killing ability of BBZ CAR-T cell and BBZ-2A-OX40 CAR-T cell, BBZ-2A-CD27 CAR-T cell and BBZ-2A-ICOS CAR-T cell in an embodiment of the present invention;

FIG. 14 is a schematic view showing the number of BBZ CAR-T cell and BBZ-2A-OX40 CAR-T cell in the bone marrow in an embodiment of the present invention;

FIG. 15 is a schematic view showing the survival ability of mouse model administrated BBZ CAR-T cell and BBZ-2A-OX40 CAR-T cell in an embodiment of the present invention;

FIG. 16 is a schematic view showing the amplification ability of BBZ CAR-T cell and BBZ-2A-OX40 CAR-T cell in an embodiment of the present invention;

FIG. 17 is a schematic view showing the tumor killing ability of BBZ CAR-T cell and BBZ-2A-OX40 CAR-T cell in an embodiment of the present invention;

FIG. 18 is a schematic view showing the in vivo anti-tumor ability of BBZ CAR-T cell and BBZ-2A-OX40 CAR-T cell in an embodiment of the present invention.

### DETAILED DESCRIPTION OF THE INVENTION

### Terms

To make the disclosure easier to understand, some terms are firstly defined. As used in this application, unless expressly stated otherwise herein, each of the following terms shall have the meanings given below. Other definitions are set forth throughout the application.

As used herein, the term “about” may refer to a value or composition within an acceptable error range for a particular value or composition as determined by those skilled in the art, which will depend in part on how the value or composition is measured or determined.

As used herein, the term “administering” refers to the physical introduction of a product of the invention into a subject using any one of various methods and delivery systems known to those skilled in the art, including intravenous, intramuscular, subcutaneous, intraperitoneal, spinal or other parenteral administration, such as by injection or infusion.

As used herein, the term “antibody” (Ab) may comprise, but is not limited to, an immunoglobulin that specifically binds an antigen and contains at least two heavy (H) chains and two light (L) chains linked by disulfide bonds, or an antigen binding parts thereof. Each H chain contains a heavy chain variable region (abbreviated herein as VH) and a heavy chain constant region. The heavy chain constant region contains three constant domains, CH1 CH2, and CH3. Each light chain contains a light chain variable region (abbreviated herein as VL) and a light chain constant region. The light chain constant region contains a constant domain CL. The VH and VL regions can be further subdivided into hypervariable regions called complementarity determining regions (CDR), which are interspersed within more conservative regions called framework regions (FR). Each VH and VL contains three CDRs and four FRs, which are arranged from amino terminal to carboxy terminal in the following order: FR1, CDR1, FR2, CDR2, FR3, CDR3, and FR4. The variable regions of the heavy and light chains contain a binding domain that interacts with an antigen.

As used herein, the term “Single-chain Fv” also abbreviated as “scFv,” are antibody fragments that comprise the VH and VL antibody domains connected into a single polypeptide chain. In some embodiments, the scFv polypeptide further comprises a polypeptide linker between the VH and VL domains which enables the scFv to form the desired structure for antigen binding. For a review of scFv, please refer to Plückthun in The Pharmacology of Monoclonal Antibodies, vol. 113, Rosenburg and Moore eds., Springer-Verlag, New York, pp. 269-315 (1994).

As used herein, the term “chimeric antigen receptor (CAR)” generally refers to an antigen receptor fused by fusing an antigen binding region of an antibody which recognizes a tumor associated antigen (TAA) or a binding fragment of other target molecules with an “immune receptor tyrosine-based activation motifs (ITAM, typically CD3ζ or FccRIγ) of an intracellular signal domain. For example, the basic structure of CAR can include an antigen binding domain of a tumor-associated antigen (TAA) or other target molecules (typically, an scFv originated from the antigen binding region of a monoclonal antibody), an extracellular hinge region, a transmembrane region, and an immunoreceptor tyrosine-based activation motif (ITAM) of an intracellular immune receptor.

As used herein, the term “binding domain” generally refers to a domain that (specifically) binds to a given target epitope or a given target site of a target molecule (e.g., an antigen), interacts with the given target epitope or the given target site, or recognizes the given target epitope or the given target site.

As used herein, the term “specific binding” generally refers to a measurable and reproducible interaction, such as, the binding between a target and an antibody, which can determine the presence of a target in the presence of heterogeneous populations of molecules (including biomolecules). For example, antibodies that specifically bind to targets (which can be epitopes) are antibodies that bind the target(s) with greater compatibility, affinity, easiness, and/or duration than other targets. In some embodiments, the antibody specifically binds to an epitope on a protein that is conserved in proteins of different species. In another embodiment, the specific binding includes but is not limited to exclusive binding.

As used herein, the term “transmembrane domain” generally refers to a polypeptide or protein which is encoded at a DNA level by an exon including at least an extracellular region, a transmembrane region, and an intracellular region. The transmembrane domain generally includes three different structural regions: N-terminal extracellular region, middle conserved transmembrane extension region, and C-terminal cytoplasmic region. The transmembrane domain may further include an intracellular region or a cytoplasmic region.

As used herein, the term “hinge region” generally refers to a region located between the binding domain and the transmembrane domain in the CAR structure. The hinge region usually comes from IgG family, such as IgG1 and IgG4, and some from IgD and CD8. Generally, the hinge region has a certain degree of flexibility, which affects the spatial constraints between the CAR molecule and its specific target, thereby affecting the contact between CAR T cells and tumor cells.

As used herein, the term “costimulatory” generally refers to a source of the second signal of lymphocyte activation, which is usually generated by an interaction of costimulatory molecules on the surface of immune cells (between T cells/B cells or between antigen presenting cells/T cells) involved in adaptive immunity with their receptors. For example, the complete activation of T cells depends on dual signaling and the action of cytokine. The first signal of T cell activation is derived from the specific binding of its receptors with the antigens, that is, the recognition of T cells to the antigens; and the second signal of T cell activation is derived from the costimulatory molecule, that is, the interaction of the costimulatory molecules of the antigen presenting cells with the corresponding receptors on the surfaces of T cells.

As used herein, the term “costimulatory domain” generally refers to an intracellular portion of the corresponding receptor of the costimulatory molecule, which can transduce a costimulatory signal (also known as the second signal). For example, in CAR-T cells, the costimulatory domain derived from CD137 (or receptors of other costimulatory molecules) can be activated after the binding of the extracellular binding domain in the CAR structure with the corresponding antigen, thereby transducing a costimulatory signal.

As used herein, the term “primary signal transduction domain” generally refers to an amino acid sequence within a cell that can generate signals which promote the immune effector function of CAR-containing cells such as CAR-T cells. Examples of the immune effector functions in, e.g., CAR-T cells can include cell lysis activity and auxiliary activity, including cytokine secretion. In some embodiments, the primary signal transduction domain transduces the effector functional signals and directs the cells to perform the specialization function. Although the primary signal transduction domain can be used in its entirety, it is not necessary to use the entire chain in many cases. As for the use of a truncated portion of the primary signal transduction domain, such truncated portion can be used to replace the intact chain, as long as it can transduce the effector functional signals. The term “primary signal transduction domain” is thus intended to encompass any truncated portion of an intracellular signal transduction domain that is sufficient to transduce the effector functional signals. For example, in CAR-T cells, the primary signal transduction domain derived from CD3zeta.

As used herein, the term “tumor” generally refers to a neoplasm or solid lesion formed by abnormal cell growth. In the present application, the tumor can be a solid tumor or a non-solid tumor. In some embodiments, a visible lump that can be detected by clinical examinations such as, X-ray radiography, CT scanning, B-ultrasound or palpation can be called solid tumor, while a tumor that cannot be seen or touched by X-ray, CT scanning, B-ultrasound and palpation, such as leukemia, can be called non-solid tumor.

As used herein, the term “pharmaceutically acceptable diluent” or “pharmaceutically acceptable excipient” generally refers to a pharmaceutically acceptable substance, composition, or vehicle involved in carrying, storing, transferring, or administering a cell preparation, e.g., liquids, semi-solid or solid fillers, diluents, osmotic agents, solvent, or encapsulating substances. The pharmaceutically acceptable diluent or excipient can include a pharmaceutically acceptable salt, wherein the term “pharmaceutically acceptable salt” includes salts of active compounds prepared by using a relatively nontoxic acid or base, e.g., sodium chloride, depending on the cell nature of the present application. The pharmaceutically acceptable carrier can further include organic acids (e.g., lactic acid), bioactive substances (e.g., polypeptides, antibodies, and the like) and antibiotics (e.g., penicillin, streptomycin), etc. The pharmaceutically acceptable carrier can further include a hydrogel, such as, a hydrogel containing polyacrylamide. The pharmaceutically acceptable diluent or excipient can include storage solution, cryopreservation solution, injection, etc., which can be used for cells. In general, the pharmaceutically acceptable diluent or excipient can maintain the activity of the cells carried by the carrier without hindering its therapeutic efficacy. The pharmaceutically acceptable diluent or excipient can also contribute to the storage, transportation, proliferation and migration of cells, and is suitable for clinical application.

As used herein, the term “subject” generally refers to a human or non-human animal, including but not limited to a cat, dog, horse, pig, cow, sheep, rabbit, mouse, rat, or monkey. In some embodiments, said subject is a human.

As used herein, the term “include/including” or “comprise/comprising” generally refers to encompassing clearly specified features, but does not exclude other elements.

The present invention provides a chimeric antigen receptor including a co-stimulatory receptor having a structure of scFv(X)-(Y)CD3zeta-2A-(Z); wherein X is a tumor-targeting antibody or other protein; Y is an intracellular domain of a co-stimulatory receptor, and said co-stimulatory receptor is selected from a group consisting of ICOS, CD28, CD27, HVEM, LIGHT, CD40L, 4-1BB, OX40, DR3, GITR, CD30, TIM1, SLAM, CD2, and CD226; Z is a co-stimulatory receptor, and said co-stimulatory receptor is a group consisting of selected from ICOS, CD28, CD27, HVEM, LIGHT, CD40L, 4-1BB, OX40, DR3, GITR, CD30, TIM1, SLAM, CD2, and CD226. The present invention also relates to a CAR-T cell constructed by a recombinant expression vector of any one of the aforesaid chimeric antigen receptor and a preparation method therefor, a formulation including the CAR-T cell, and a use of the CAR-T cell.

Hereinafter the embodiments of the present invention are further described with reference to the accompanying drawings and examples. The following examples are only for more clearly illustrating the technical solutions of the present invention, but not for limiting the protective scope of the present invention.

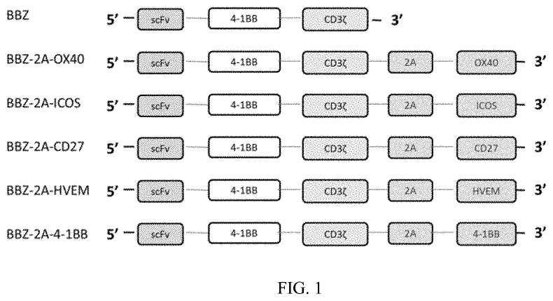

The chimeric antigen receptor (CAR) molecules including a co-stimulatory receptor used in the following examples of the present invention are BBZ-2A-OX40, BBZ-2A-HVEM, BBZ-2A-ICOS, BBZ-2A-CD27, and BBZ-2A-4-1BB, respectively, and their structures are shown in FIG. 1.

### Example 1—Preparation of 20BBZ-2A-OX40 CAR-T Cell

The preparation of the 20BBZ-2A-OX40 CAR-T cell in this example includes the following steps:

1. Construction of lentiviral vector pCDH-MSCVEF-20BBZ-2A-OX40 and production of virus

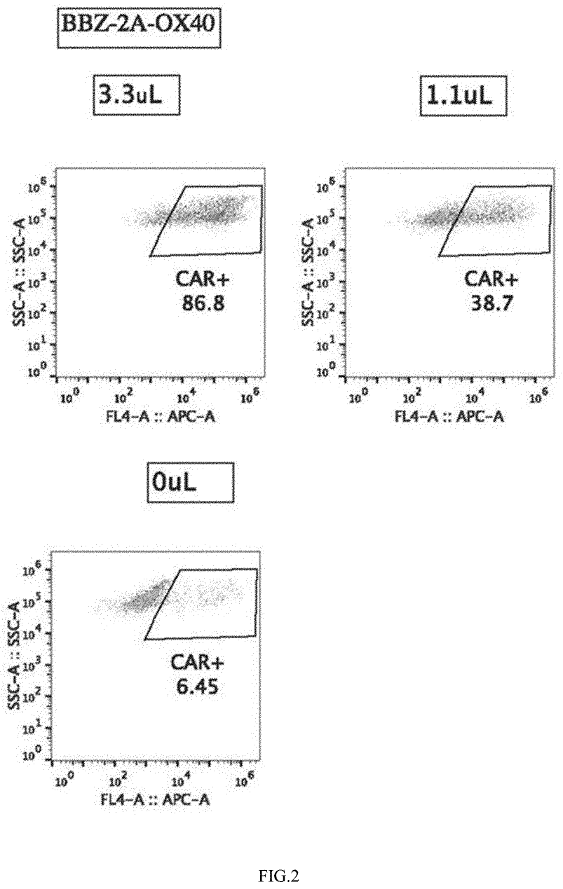

incorporating 2A (SEQ ID No. 7) sequence between scFv-antihCD20-20BBZ (SEQ ID No. 1) and OX40 (SEQ ID No.2) by overlap PCR, and adding EcoRI and SalI restriction sites to both ends to clone the pCDH-MSCVEF vector. Subjecting the clones sequenced correctly to a large scale endotoxin-free extraction, and co-transfecting with lentiviral packaging plasmid (VSV-g, pMD Gag/Pol, RSV-REV) into 293X. After 48 and 72 hours, collecting the supernatant, filtering it by a 0.45 μm filter, and performing centrifugation with Beckman ultracentrifuge and SW28 head at 25000 RPM for 2 hours to concentrate the virus, which is pCDH-MSCVEF-20BBZ-2A-OX40 virus (briefly, 20BBZ-2A-OX40 virus) for the subsequent production of CAR-T cell. Meanwhile, producing the control pCDH-MSCVEF-20BBZ virus (briefly, 20BBZ virus), and infecting 293 cells with the obtained virus to measure the virus titer, as shown in FIG. 2.

2. Preparation of 20BBZ-2A-OX40 CAR-T cell and 20BBZ CAR-T cell

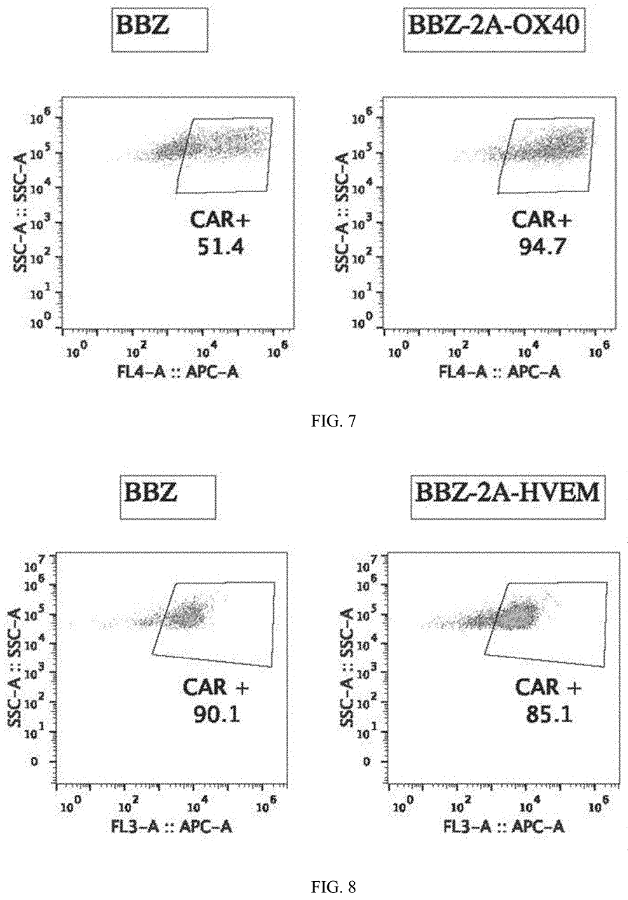

purifying human PBMC by a Stemcell T cell isolation kit, and inoculating into a 96-well culture plate coated with anti-hCD3 and anti-hCD28 antibody. After 2 days, infecting the cells with 20BBZ virus and 20BBZ-2A-OX40 virus at MOI=10-20. After 1 day, continuing to culture the cells with the medium changed, and stimulating them by artificial antigen presenting cell or anti-hCD3/28 antibody every 6 days. After 2 rounds of stimulation, the obtained cells are 20BBZCAR-T cell and 20BBZ-2A-OX40 CAR-T cell for subsequent experiments and phenotypic analysis. The results are shown in FIG. 7. It can be seen that the obtained cells are CAR-POSITIVE.

### Example 2—Preparation of 20BBZ-2A-HVEM CAR-T Cell

The preparation of the 20BBZ-2A-HVEM CAR-T cell in in this example includes the following steps:

1. Construction of lentiviral vector pCDH-MSCVEF-20BBZ-2A-HVEM and production of virus

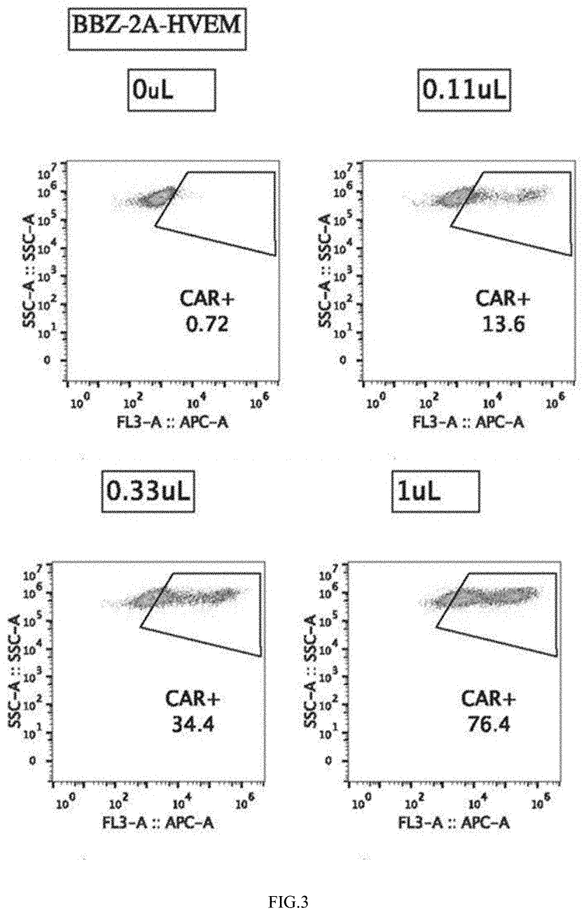

incorporating 2A (SEQ ID No. 8) sequence between scFv-antihCD20-20BBZ (SEQ ID No.1) and HVEM (SEQ ID No.3) by overlap PCR, and adding EcoRI and SalI restriction sites to both ends to clone pCDH-MSCVEF vector. Subjecting the clones sequenced correctly to a large scale endotoxin-free extraction, and co-transfecting with lentiviral packaging plasmid (VSV-g, pMD Gag/Pol, RSV-REV) into 293X. After 48 and 72 hours, collecting the supernatant, filtering it by a 0.45 μm filter, and performing centrifugation with Beckman ultracentrifuge and SW28 head at 25000 RPM for 2 hours to concentrate the virus, which is pCDH-MSCVEF-20BBZ-2A-HVEM virus (briefly, 20BBZ-2A-HVEM virus) for the subsequent production of CAR-T cell. Meanwhile, producing the control pCDH-MSCVEF-20BBZ virus (briefly, 20BBZ virus). Infecting 293 cells with the obtained virus to measure the virus titer, as shown in FIG. 3.

2. Preparation of 20BBZ-2A-HVEM CAR-T cell and 20BBZ CAR-T cell

purifying human PBMC by a Stemcell T cell isolation kit, and inoculating into a 96-well culture plate coated with anti-hCD3 and anti-hCD28 antibody. After 2 days, infecting the cells were infected with 20BBZ virus and 20BBZ-2A-HVEM virus at MOI=10-20. After 1 day, continuing to culture the cells with the medium changed, and stimulating them by artificial antigen presenting cell or anti-hCD3/28 antibody every 6 days. After 2 rounds of stimulation, the obtained cells are 20BBZCAR-T cell and 20BBZ-2A-HVEM CAR-T cell for subsequent experiments and phenotypic analysis. The results are shown in FIG. 8. It can be seen from the figure that the obtained cells are CAR-POSITIVE.

### Example 3—Preparation of 20BBZ-2A-ICOS CAR-T Cell

The preparation of the 20BBZ-2A-ICOS CAR-T cell in this example includes the following steps:

1. Construction of lentiviral vector pCDH-MSCVEF-20BBZ-2A-ICOS and production of virus

incorporating 2A (SEQ ID No. 9) sequence between scFv-antihCD20-20BBZ (SEQ ID No.1) and ICOS (SEQ ID No.4) by overlap PCR, and adding EcoRI and SalI restriction sites to both ends to clone pCDH-MSCVEF vector. Subjecting the clones sequenced correctly to a large scale endotoxin-free extraction, and co-transfecting with lentiviral packaging plasmid (VSV-g, pMD Gag/Pol, RSV-REV) into 293X. After 48 and 72 hours, collecting the supernatant, filtering it by a 0.45 μm filter, and performing centrifugation with Beckman ultracentrifuge and SW28 head at 25000 RPM for 2 hours to concentrate the virus, which is pCDH-MSCVEF-20BBZ-2A-ICOS virus (briefly, 20BBZ-2A-ICOS virus) for the subsequent production of CAR-T cell. Meanwhile, producing the control pCDH-MSCVEF-20BBZ virus (briefly, 20BBZ virus), and infecting 293 cells with the obtained virus to measure the virus titer, as shown in FIG. 4.

2. Preparation of 20BBZ-2A-ICOS CAR-T cell and 20BBZ CAR-T cell

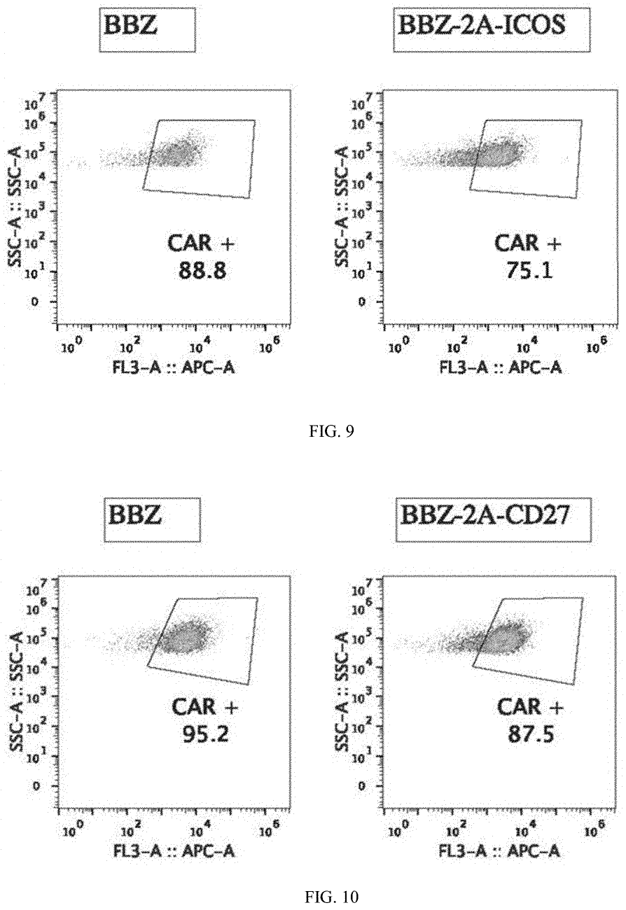

purifying human PBMCs by a Stemcell T cell isolation kit, and inoculating into a 96-well culture plate coated with anti-hCD3 and anti-hCD28 antibody. After 2 days, infecting the cells with 20BBZ virus and 20BBZ-2A-ICOS virus at MOI=10-20. After 1 day, continuing to culture the cells with the medium changed, and stimulating them by artificial antigen presenting cell or anti-hCD3/28 antibody every 6 days. After 2 rounds of stimulation, the obtained cells are 20BBZCAR-T cell and 20BBZ-2A-ICOS CAR-T cell for subsequent experiments and phenotypic analysis. The results are shown in FIG. 9. It can be seen from the figure that the obtained cells are CAR-POSITIVE.

### Example 4—Preparation of 20BBZ-2A-CD27 CAR-T Cell

The preparation of 20BBZ-2A-CD27 CAR-T cell in this example includes the following steps:

1. Construction of lentiviral vector pCDH-MSCVEF-20BBZ-2A-CD27 and production of virus

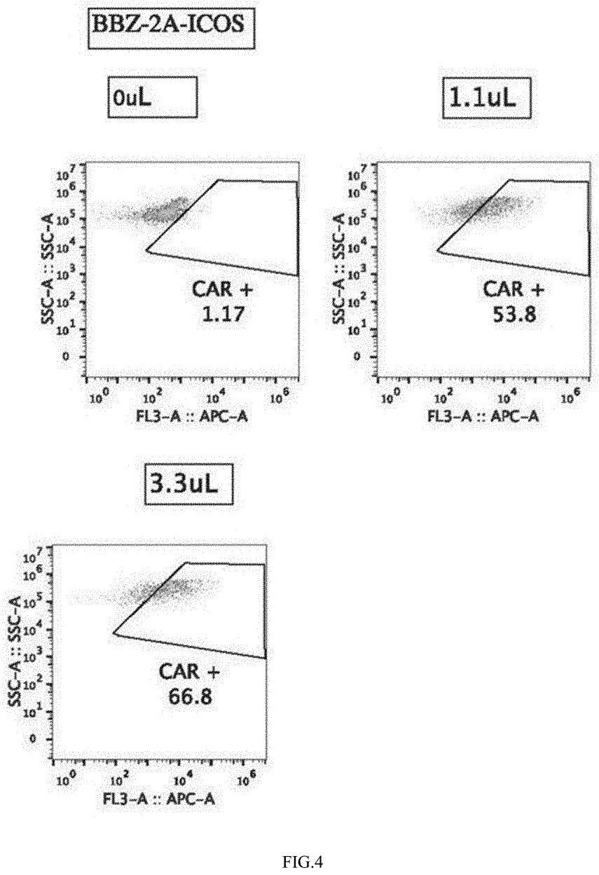

incorporating 2A (SEQ ID No. 10) sequence between scFv-antihCD20-20BBZ (SEQ ID No.1) and CD27 (SEQ ID No.5) by overlap PCR, and adding EcoRI and SalI restriction sites to both ends to clone pCDH-MSCVEF vector. Subjecting the clones sequenced correctly to a large scale endotoxin-free extraction, and co-transfecting with lentiviral packaging plasmid (VSV-g, pMD Gag/Pol, RSV-REV) into 293X. After 48 and 72 hours, collecting the supernatant, filtering it by a 0.45 μm filter, and performing centrifugation with Beckman ultracentrifuge and SW28 head at 25000 RPM for 2 hours to concentrate the virus, which is pCDH-MSCVEF-20BBZ-2A-CD27 virus (briefly, 20BBZ-2A-CD27 virus) for the subsequent production of CAR-T cell. Meanwhile, producing the control pCDH-MSCVEF-20BBZ virus (briefly, 20BBZ virus), and infecting 293 cells with the obtained virus to measure the virus titer, as shown in FIG. 5.

2. Preparation of 20BBZ-2A-CD27 CAR-T cell and 20BBZ CAR-T cell

purifying human PBMC by a Stemcell T cell isolation kit, and inoculating into a 96-well culture plate coated with anti-hCD3 and anti-hCD28 antibody. After 2 days, infecting the cells with 20BBZ virus and 20BBZ-2A-CD27 virus at MOI=10-20. After 1 day, continuing to culture the cells with the medium changed, and stimulating them by artificial antigen presenting cell or anti-hCD3/28 antibody every 6 days. After 2 rounds of stimulation, the obtained cells are 20BBZCAR-T cell and 20BBZ-2A-CD27 CAR-T cell for subsequent experiments and phenotypic analysis. The results are shown in FIG. 10. It can be seen from the figure that the obtained cells are CAR-POSITIVE.

### Example 5—Preparation of 20BBZ-2A-4-1BB CAR-T Cell

The preparation of the 20BBZ-2A-4-1BB CAR-T cell in this example includes the following steps:

1. Construction of lentiviral vector pCDH-MSCVEF-20BBZ-2A-4-1BB and production of virus

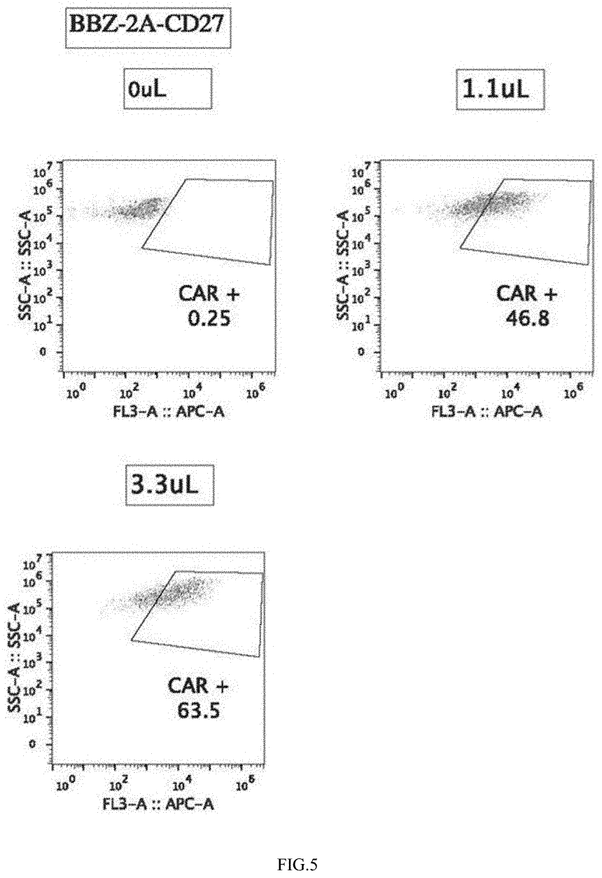

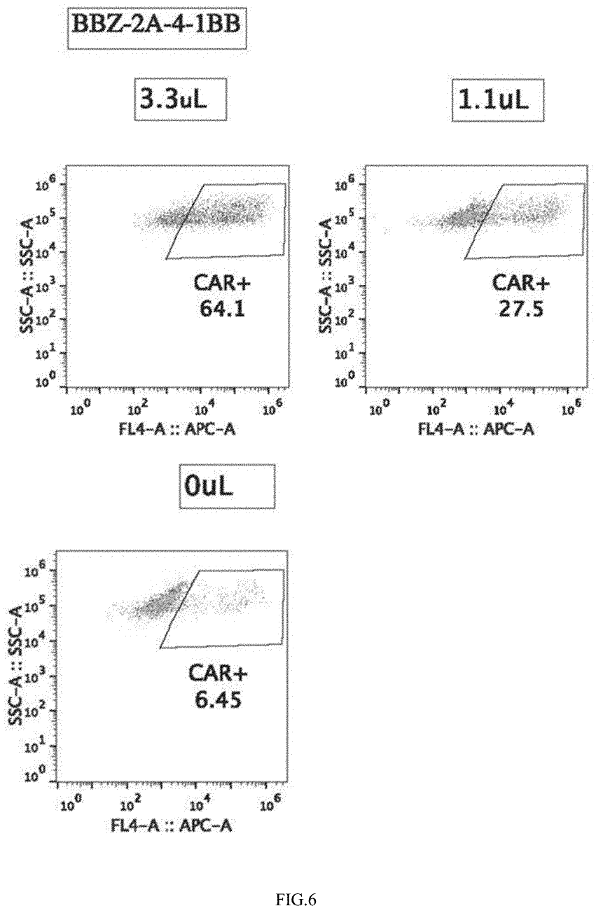

incorporating 2A (SEQ ID No. 7) sequence between scFv-antihCD20-20BBZ (SEQ ID No.1) and 4-1BB (SEQ ID No.6) by overlap PCR, and adding EcoRI and SalI restriction sites to both ends to clone pCDH-MSCVEF vector. Subjecting the clones sequenced correctly to a large scale endotoxin-free extraction, and co-transfecting with lentiviral packaging plasmid (VSV-g, pMD Gag/Pol, RSV-REV) into 293X. After 48 and 72 hours, collecting the supernatant, filtering it by a 0.45 μm filter, and performing centrifugation with Beckman ultracentrifuge and SW28 head at 25000 RPM for 2 hours to concentrate the virus, which is pCDH-MSCVEF-20BBZ-2A-4-1BB virus (briefly, 20BBZ-2A-4-1BB virus) for the subsequent production of CAR-T cell. Meanwhile, producing the control pCDH-MSCVEF-20BBZ virus (briefly, 20BBZ virus), infecting 293 cells with the obtained virus to measure the virus titer, as shown in FIG. 6.

2. Preparation of 20BBZ-2A-4-1BB CAR-T cell and 20BBZ CAR-T cell

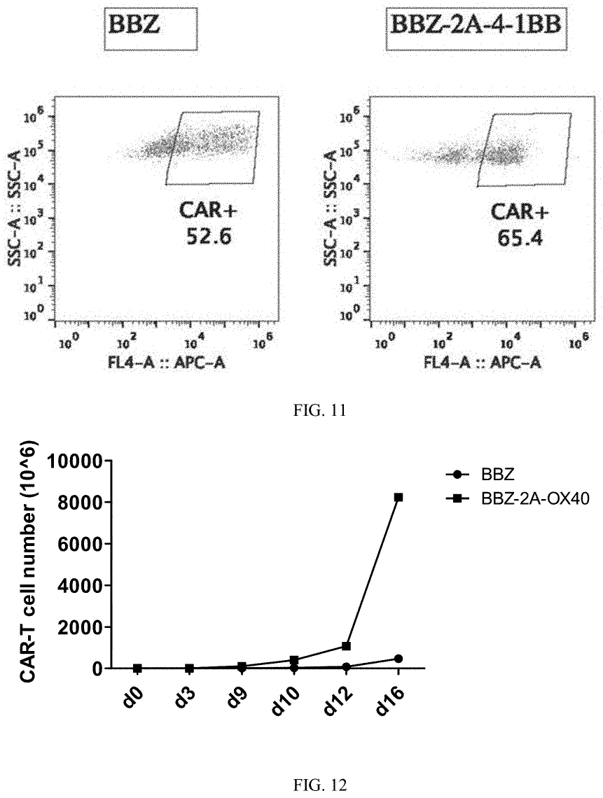

purifying human PBMC by a Stemcell T cell isolation kit, and inoculating into a 96-well culture plate coated with anti-hCD3 and anti-hCD28 antibody. After 2 days, infecting the cells with 20BBZ virus and 20BBZ-2A-4-1BB virus at MOI=10-20. After 1 day, continuing to culture the cells with the medium changed, and stimulating them by artificial antigen presenting cell or anti-hCD3/28 antibody every 6 days. After 2 rounds of stimulation, the obtained cells are 20BBZCAR-T cell and 20BBZ-2A-4-1BB CAR-T cell for subsequent experiments and phenotypic analysis. The results are shown in FIG. 11. It can be seen from the figure that the obtained cells are CAR-POSITIVE.

### Example 6—Comparison of Expansion Abilities of 20BBZ CAR-T Cell and 20BBZ-2A-OX40 CAR-T Cell

20BBZ CAR-T cell and 20BBZ-2A-OX40 CAR-T cell prepared in Step 2 of Example 1 were continuously cultured for 14 days (as the 20BBZ CAR-T cell and 20BBZ-2A-OX40 CAR-T were obtained on day 2 in FIG. 12), and stimulated with artificial antigen presenting cell once every 6 days. The cells were counted, and the results are shown in FIG. 12. It can be seen from the figure that 20BBZ-2A-OX40 CAR-T cell has enhanced proliferation ability as compared with 20BBZCAR-T cell.

### Example 7—Comparison of Tumor-Killing Abilities of 20BBZ CAR-T Cell and 20BBZ-2A-OX40 CAR-T Cell

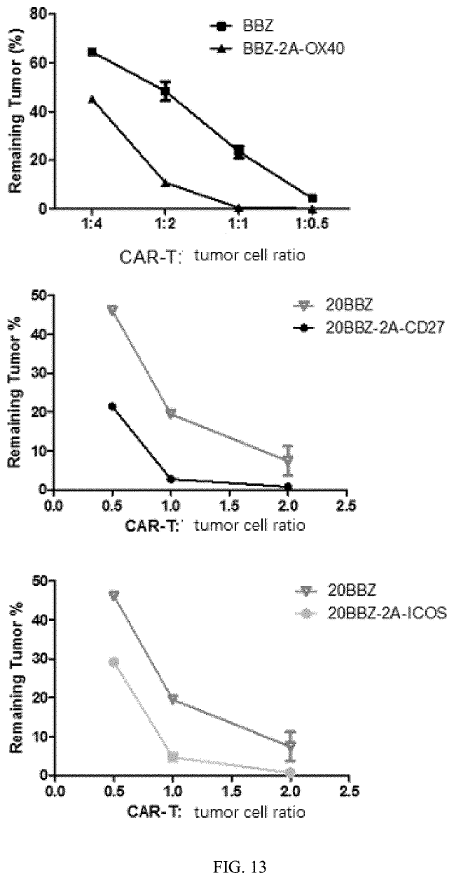

20BBZ CAR-T cell and 20BBZ-2A-OX40 CAR-T cell obtained in Step 2 of Example 1, 2OBBZ-2A-ICOS CAR-T cell obtained in Step 2 of Example 3, and 20BBZ-2A-CD27 CAR-T cell obtained in Step 2 of Example 4 were inoculated into a 96-well plate. For 20BBZ CAR-T cell and 20BBZ-2A-OX40 CAR-T cell, Raji tumor cells were added at a CAR-T: tumor cell ratio of 1:0.5, 1:1, 1:2, 1:4; For 20BBZ CAR-T cell and 20BBZ-2A-CD27 CAR-T cell, Raji tumor cells were added at a CAR-T:tumor cell ratio of 1:0.5, 1:1, 1:1.5, and 1:2; For 20BBZ CAR-T cell and 20BBZ-2A-ICOS CAR-T cell, Raji tumor cells were added at a CAR-T:tumor cell ratio of 1:0.5, 1:1, 1:1.5, and 1:2. After 24 hours, the survival rates of tumor cells were compared, and the results are shown in FIG. 13. It can be seen from the figure that 20BBZ-2A-OX40/ICOS/CD27 CAR-T cell has similar tumor killing ability as compared with 20BBZ CAR-T cell, and some CAR-T including the co-stimulatory receptor has a stronger tumor killing ability.

### Example 8—Comparison of Anti-Tumor Ability and In Vivo Survival Ability of 20BBZ CAR-T Cell and 20BBZ-2A-OX40 CAR-T Cell

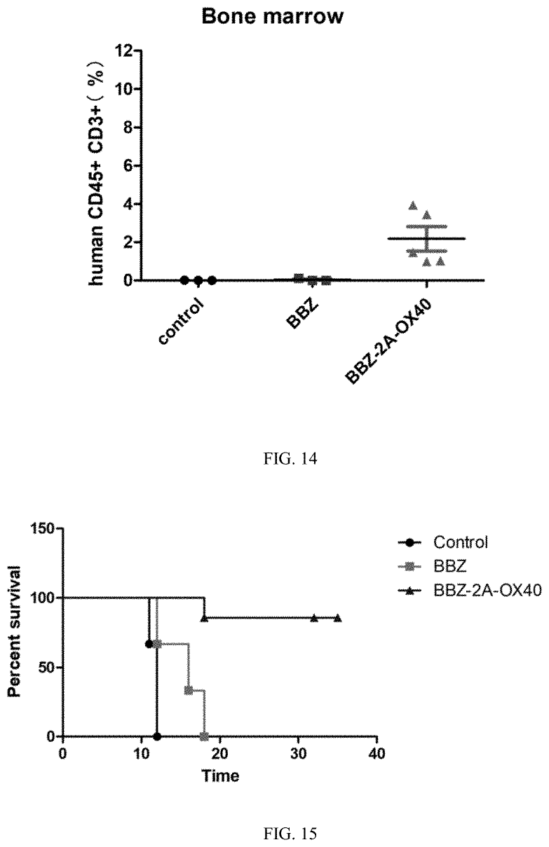

10⁶ Nalm-6 tumor cells were intravenously inoculated into B-NDG mice, which were treated with 10⁷ 2 OBBZ CAR-T cells and 20BBZ-2A-OX40 CAR-T cells after 6 days. The mice were observed for their survival rates, and some mice were detected for the level of tumor cells and CAR-T cells in their marrow on Day 7. The results are shown in FIG. 14 and FIG. 15, respectively. It can be seen from the figure that 20BBZ-2A-OX40 CAR-T cell, as compared with 20BBZ CAR-T cell, significantly prolongs the survival of mice, and expanded more in vivo.

It can be seen from the aforesaid examples that the present invention constructs a novel CAR-T cell including a co-stimulatory receptor, which significantly increases the activation ability, survival ability, expansion ability of the CAR-T cells in tumors, as compared with the current CAR-T technology in clinic use, and has a more superior anti-tumor therapeutic effect.

### Example 9 Preparation of 19BBZ-2A-OX40 CAR-T Cell

The preparation of the 19BBZ-2A-OX40 CAR-T cell in this example includes the following steps:

1. Construction of lentiviral vector pCDH-MSCVEF-19BBZ-2A-OX40 and production of virus

Incorporating 2A (SEQ ID No. 7 of US2021/0169932A1) sequence between scFv-anti-hCD19-BBZ (SEQ ID No.11) and OX40 (SEQ ID No.2) by overlap PCR, and adding EcoRI and SalI restriction sites to both ends to clone the pCDH-MSCVEF vector. Subjecting the clones sequenced correctly to a large-scale endotoxin-free extraction, and co-transfecting with lentiviral packaging plasmid (VSV-g, pMD Gag/Pol, RSV-REV) into 293X. After 48 and 72 hours, collecting the supernatant, filtering it by a 0.45 μm filter, and performing centrifugation with Beckman ultracentrifuge and SW28 head at 25000 RPM for 2 hours to concentrate the virus, which is pCDH-MSCVEF-19BBZ-2A-OX40 virus (briefly, 19BBZ-2A-OX40 virus) for the subsequent production of CAR-T cell. Meanwhile, producing the control pCDH-MSCVEF-19BBZ virus (briefly, 19BBZ virus), and infecting 293 cells with the obtained virus to measure the virus titer.

2. Preparation of 19BBZ-2A-OX40 CAR-T cell and 19BBZ CAR-T cell

purifying human PBMC by a Stemcell T cell isolation kit, and inoculating into a 96-well culture plate coated with anti-hCD3 and anti-hCD28 antibody. After 2 days, infecting the cells with 19BBZ virus and 19BBZ-2A-OX40 virus at MOI=10-20. After 1 day, continuing to culture the cells with the medium changed, and stimulating them by artificial antigen presenting cell or anti-hCD3/28 antibody every 6 days. After 2 rounds of stimulation, the obtained cells are 19BBZCAR-T cell and 19BBZ-2A-OX40 CAR-T cell for subsequent experiments and phenotypic analysis.

### Example 10 Preparation of EGFRBBZ-2A-OX40 CAR-T Cell

The preparation of the EGFRBBZ-2A-OX40 CAR-T cell in this example includes the following steps:

1. Construction of lentiviral vector pCDH-MSCVEF-EGFRBBZ-2A-OX40 and production of virus

incorporating 2A (SEQ ID No. 7 of US2021/0169932A1) sequence between scFv-anti-hEGFR-BBZ (SEQ ID No.12) and OX40 (SEQ ID No.2 of US2021/0169932A1) by overlap PCR, and adding EcoRI and SalI restriction sites to both ends to clone the pCDH-MSCVEF vector. Subjecting the clones sequenced correctly to a large scale endotoxin-free extraction, and co-transfecting with lentiviral packaging plasmid (VSV-g, pMD Gag/Pol, RSV-REV) into 293X. After 48 and 72 hours, collecting the supernatant, filtering it by a 0.45 μm filter, and performing centrifugation with Beckman ultracentrifuge and SW28 head at 25000 RPM for 2 hours to concentrate the virus, which is pCDH-MSCVEF-EGFRBBZ-2A-OX40 virus (briefly, EGFRBBZ-2A-OX40 virus) for the subsequent production of CAR-T cell. Meanwhile, producing the control pCDH-MSCVEF-EGFRBBZ virus (briefly, EGFRBBZ virus), and infecting 293 cells with the obtained virus to measure the virus titer.

2. Preparation of EGFRBBZ-2A-OX40 CAR-T cell and EGFRBBZ CAR-T cell

purifying human PBMC by a Stemcell T cell isolation kit, and inoculating into a 96-well culture plate coated with anti-hCD3 and anti-hCD28 antibody. After 2 days, infecting the cells with EGFRBBZ virus and EGFRBBZ-2A-OX40 virus at MOI=10-20. After 1 day, continuing to culture the cells with the medium changed, and stimulating them by artificial antigen presenting cell or anti-hCD3/28 antibody every 6 days. After 2 rounds of stimulation, the obtained cells are EGFRBBZCAR-T cell and EGFRBBZ-2A-OX40 CAR-T cell for subsequent experiments and phenotypic analysis.

### Example 11 Comparison of Expansion Abilities of 19BBZ CAR-T Cell and 19BBZ-2A-OX40 CAR-T Cell

19BBZ CAR-T cell and 19BBZ-2A-OX40 CAR-T cell prepared in Step 2 of Example 2 were continuously cultured for 18 days (as the 19BBZ CAR-T cell and 19BBZ-2A-OX40 CAR-T cell were obtained on day 2 in FIG. 12), and stimulated with artificial antigen presenting cell once every 6 days. The cells were counted, and the results are shown in FIG. 16. It can be seen from the figure that 19BBZ-2A-OX40 CAR-T cell has enhanced proliferation ability as compared with 19BBZCAR-T cell.

### Example 12 Comparison of Tumor-Killing Abilities of 19BBZ CAR-T Cell and 19BBZ-2A-OX40 CAR-T Cell

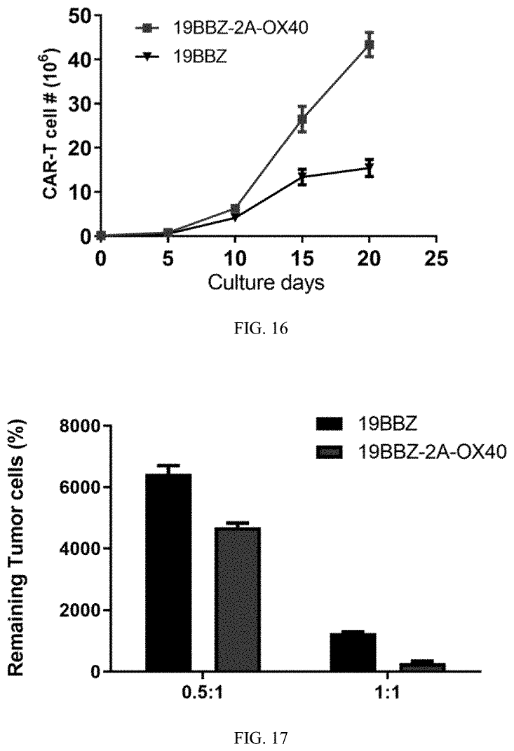

19BBZ CAR-T cell and 19BBZ-2A-OX40 CAR-T cell obtained in Step 2 of Example 2 were inoculated into a 96-well plate, and Raji tumor cells were added at a CAR-T:tumor cell ratio of 1:1, and 0.5:1. After 24 hours, the survival rates of tumor cells were compared, and the results are shown in FIG. 17. It can be seen from the figure that 19BBZ-2A-OX40 has stronger tumor killing ability as compared with 19BBZ CAR-T cell.

### Example 13 Comparison of Anti-Tumor Ability In Vivo of EGFRBBZ CAR-T Cell and EGFRBBZ-2A-OX40 CAR-T Cell

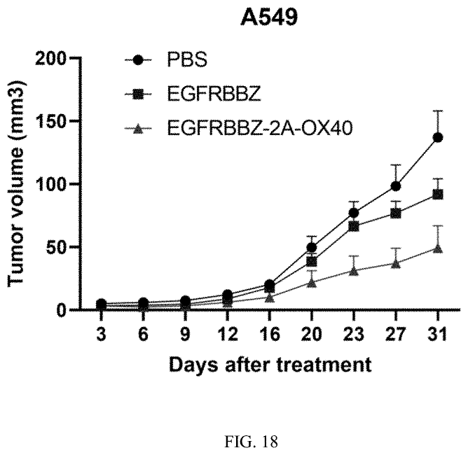

10⁶ A549 tumor cells were subcutaneously inoculated into on the right flank of B-NDG mice, which were treated with 10⁷ EGFRBBZ CAR-T cells and EGFRBBZ-2A-OX40 CAR-T cells after 6 days. Tumor volumes were measured twice a week along three orthogonal axes (length, width, and height) and tumor volumes calculated using the equation (length×width×height)/2. The results are shown in FIG. 18. It can be seen from the figure that EGFRBBZ-2A-OX40 CAR-T cell, as compared with EGFRBBZ CAR-T cell, significantly reduced the tumor volumes in mice, indicating enhanced antitumor activity in vivo.

Hereinbefore the specific embodiments of the present invention are described in details. However, they are only used as examples, and the present invention is not limited to the specific embodiments as described above. For those skilled in the art, any equivalent modifications and substitutions made to the present invention are encompassed in the scope of the present invention. Therefore, all the equal transformations and modifications without departing from the spirit and scope of the present invention should be covered in the scope of the present invention.

What is claimed is:

1. A chimeric antigen receptor comprising a co-stimulatory receptor, wherein said chimeric antigen receptor has a structure of scFv(X)-(Y)CD3zeta-2A-(Z);
wherein X comprises a tumor-targeting antibody or a ligand or receptor capable of specifically binding to a tumor;
Y is an intracellular domain of a co-stimulatory receptor, and said co-stimulatory receptor is selected from a group consisting of ICOS, CD28, CD27, HVEM, LIGHT, CD40L, 4-1BB, OX40, DR3, GITR, CD30, TIM1, SLAM, CD2, and CD226; and
Z is a co-stimulating receptor, and said co-stimulatory receptor is selected from a group consisting of ICOS, CD28, CD27, HVEM, LIGHT, CD40L, 4-1BB, OX40, DR3, GITR, CD30, TIM1, SLAM, CD2, and CD226.

2. The chimeric antigen receptor comprising a co-stimulatory receptor according to claim 1, wherein said X is selected from a group consisting of anti-CD19 antibody, anti-CD20 antibody, anti-EGFR antibody, anti-HER2 antibody, anti-EGFRVIII antibody, anti-PSMA antibody, anti-BCMA antibody, anti-CD22 antibody, and anti-CD30 antibody.

3. The chimeric antigen receptor comprising a co-stimulatory receptor according to claim 1, wherein said X is anti-CD20 antibody, anti-CD19 antibody or EGFR antibody, said Y is 4-1BB, said Z is one selected from a group consisting of OX40, HVEM, ICOS, CD27, and 4-1BB.

4. The chimeric antigen receptor comprising a co-stimulatory receptor according to claim 3, wherein said scFv(X)-(Y)CD3zeta is anti-CD20 scFv with a sequence of SEQ ID No.1; anti-CD19 scFv with a sequence of SEQ ID No.11, and anti-EGFR scFv with a sequence of SEQ ID No.12.

5. The chimeric antigen receptor comprising a co-stimulatory receptor according to claim 3, wherein said OX40 has a sequence of SEQ ID No.2; said HVEM has a sequence of SEQ ID No.3; said ICOS has a sequence of SEQ ID No.4; said CD27 has a sequence of SEQ ID No.5; and/or, said 4-1BB has a sequence of SEQ ID No.6.

6. The chimeric antigen receptor comprising a co-stimulatory receptor according to claim 3, wherein said 2A has a sequence of SEQ ID No.7; SEQ ID No.8; SEQ ID No.9 or SEQ ID No.10.

7. A CAR-T cell constructed by a recombinant expression vector of said chimeric antigen receptor according to any one of claims 1-4.

8. A method of preparing said CAR-T cell according to claim 5, comprising the following steps:
step 1, construction of lentiviral vector and production of virus;
incorporating 2A between scFv(X)-(Y)CD3zeta and Z to form a fusion protein, adding a lentiviral vector to both ends of the fusion protein, and co-transfecting with a lentiviral packaging plasmid to obtain an scFv(X)-(Y)CD3zeta-2A-(Z) virus.

9. The method of preparing said CAR-T cell according to claim 8, wherein the method comprises the following steps:
step 2, preparation of scFv(X)-(Y)CD3zeta-2A-(Z) CAR-T cell;
culturing purified human PBMC, and infecting the T cell isolated from said PBMC with the scFv(X)-(Y)CD3zeta-2A-(Z) virus obtained in Step 1, subjecting them to cell expansion under suitable conditions to prepare the scFv(X)-(Y)CD3zeta-2A-(Z) CAR-T cell.

10. The method of preparing said CAR-T cell according to claim 8, wherein said construction of lentiviral vector and production of virus comprises:
incorporating 2A between scFv(X)-(Y)CD3zeta and Z by overlap PCR to form a fusion protein, and adding restriction sites to both ends of the fusion protein to clone a lentiviral vector;
subjecting the clones sequenced correctly to a large scale endotoxin-free extraction, and co-transfecting with a lentiviral packaging plasmid; after a predetermined time period, collecting a supernatant, filtering, centrifuging to concentrate the virus to obtain an scFv(X)-(Y)CD3zeta-2A-(Z) virus.

11. The method of preparing said CAR-T cell according to claim 8, wherein said preparation of said scFv(X)-(Y)CD3zeta-2A-(Z) CAR-T cell comprises: isolating T cells from human PBMC for purification, inoculating into a culture plate under suitable stimulation conditions, culturing them for a predetermined period of time, infecting said T cells with the scFv(X)-(Y)CD3zeta-2A-(Z) virus produced in Step 1, and subjecting them to cell expansion under suitable stimulation conditions, after 2 rounds of expansion under stimulation, the obtained cells are the scFv(X)-(Y)CD3zeta-2A-(Z) CAR-T cells.

12. A formulation, comprising said CAR-T cell according to claim 7.

13. A method of treating or preventing tumors, comprising administrating said chimeric antigen receptor according to any one of claims 1-6 or said CAR-T cell according to claim 7 to the subject in need of.

14. The method of treating or preventing tumors according to claim 13, wherein said tumor is selected from the group consisting of a hematological tumor, a solid tumor, and a combination thereof.

15. The method of treating or preventing tumors according to claim 13, wherein said tumor comprises Burkitt lymphoma (BL), acute lymphoblastic leukemia (ALL), and/or lung cancer.


SEQUENCE LISTING (12 sequences)

SEQ ID NO: 1 - protein, 468 aa, synthetic construct
  REGION 1..468: note = the sequence of scFv-antihCD20-20BBZ
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 277 aa, synthetic construct
  REGION 1..277: note = the sequence of OX40
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 3 - protein, 283 aa, synthetic construct
  REGION 1..283: note = the sequence of HVEM
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 4 - protein, 199 aa, synthetic construct
  REGION 1..199: note = the sequence of ICOS
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 5 - protein, 260 aa, synthetic construct
  REGION 1..260: note = the sequence of CD27
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCRYSCPREEEGSTIPIQEDYRKPEPACSP

SEQ ID NO: 6 - protein, 255 aa, synthetic construct
  REGION 1..255: note = the sequence of 4-1BB
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 7 - protein, 22 aa, synthetic construct
  REGION 1..22: note = the sequence of 2A
GSGATNFSLLKQAGDVEENPGP

SEQ ID NO: 8 - protein, 21 aa, synthetic construct
  REGION 1..21: note = the sequence of 2A
GSGEGRGSLLTCGDVEENPGP

SEQ ID NO: 9 - protein, 23 aa, synthetic construct
  REGION 1..23: note = the sequence of 2A
GSGQCTNYALLKLAGDVESNPGP

SEQ ID NO: 10 - protein, 25 aa, synthetic construct
  REGION 1..25: note = the sequence of 2A
GSGVKQTLNFDLLKLAGDVESNPGP

SEQ ID NO: 11 - protein, 264 aa, synthetic construct
METDTLLLWVLLLWVPGSTGTGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 12 - protein, 263 aa, synthetic construct
METDTLLLWVLLLWVPGSTGTGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA